(19) United States
(12) Patent Application Publication
(10) Pub. No.: US 2020/0155481 A1
(43) Pub. Date: May 21, 2020

(54) INTRAMUSCULAR ATOVAQUONE FOR MALARIA PROPHYLAXIS

(71) Applicant: THE JOHNS HOPKINS UNIVERSITY, Baltimore, MD (US)

(72) Inventors: Theresa A. Shapiro, Baltimore, MD (US); Rahul P. Bakshi, Baltimore, MD (US); Abhai Tripathi, Ellicott City, MD (US); Godfree Mlambo, Perry Hall, MD (US)

(21) Appl. No.: 16/610,684

(22) PCT Filed: May 3, 2018
(86) PCT No.: PCT/US2018/030764

§ 371 Date: Nov. 4, 2019

Related U.S. Application Data
(60) Provisional application No. 62/500,732, filed on May 3, 2017.

(51) Int. Cl. A61K 31/122; A61K 9/00; A61P 31/10; A61P 33/06

(52) U.S. Cl. CPC A61K 31/122; A61K 9/0019; A61K 9/10; A61P 33/06; A61P 31/10

(57) ABSTRACT

The present invention relates to the field of malaria. More specifically, the present invention provides compositions and methods useful for the prophylaxis and treatment of malaria, as well as other parasitic and fungal infections. In a specific embodiment, the present invention provides a method for preventing malaria comprising the step of intramuscularly administering an effective amount of atovaquone to a subject.

### CROSS-REFERENCE TO RELATED APPLICATIONS

This application claims the benefit of U.S. Provisional Application No. 62/500,732, filed May 3, 2017, which is incorporated herein by reference in its entirety.

### FIELD OF THE INVENTION

The present invention relates to the field of malaria. More specifically, the present invention provides compositions and methods useful for the prophylaxis and treatment of malaria, as well as other parasitic and fungal infections.

### BACKGROUND OF THE INVENTION

Malaria is one of most dangerous infectious diseases in tropical and subtropical countries, afflicting about 300 million people. The pathogen of the disease is a protozoan parasite, Plasmodium sp. which is transmitted by Anopheles mosquitoes. Four major species of malaria parasites can infect humans under natural conditions: Plasmodium falciparum, P. vivax, P. ovale, and P. malariae. P. falciparum and P. vivax cause the most infections worldwide. P. falciparum is the agent of severe, potentially fatal malaria. Malaria caused by P. falciparum is responsible for nearly 500 thousand deaths annually. Based on recent estimates from the WHO, worldwide, there were an estimated 216 million malaria cases among 3.3 billion people at risk living in 91 countries (WHO World Malaria Report 2017). Infections caused by P. falciparum and P. vivax account for more than 90% of global malaria burden; the former being responsible for nearly all the deaths due to malaria, most in children under 5 years. Malaria also poses a serious threat (including death) to tens of millions of nonimmune travellers per year who travel to malarious areas. Leder et al., 39 CLIN. INFECT. DIS. 1104-12 (2004). Malaria in travellers is commonly associated with noncompliant or no oral chemoprophylaxis. Cullen et al., 65 MMWR SURVEILL. SUMM. 1-22 (2016). Given the current lack of a clinically useful vaccine, long-acting parenteral chemoprophylaxis would provide convenient and durable antimalarial protection, effective within hours of dosing, that eliminates noncompliance and the necessity for carrying (and taking) medication.

### SUMMARY OF THE INVENTION

The present invention is based, at least in part, on the discovery that atovaquone can be administered intramuscularly and provide chemoprophylaxis against malaria. GlaxoSmithKline's Mepron® (atovaquone) is used to treat various opportunistic and parasitic infections. In the murine Plasmodium berghei sporozoite challenge model, the present inventors found that orally dosed Mepron fails by one week after dosing, but the same amount dosed intramuscularly protects for five weeks. Thus, the present invention provides compositions and methods related to the intramuscular injection of atovaquone.

In a specific embodiment, a method for preventing malaria comprises the step of intramuscularly administering an effective amount of atovaquone to a subject. In a more specific embodiment, the atovaquone is present as a microparticle. The atovaquone can be administered every few weeks including, but not limited to, every two weeks, every three weeks, every four weeks, every five weeks, every six weeks, every seven weeks, every 8 weeks and so forth. In a specific embodiment, atovaquone can be administered once every 2-8 weeks. In more specific embodiments, atovaquone can be administered once every 2-3 weeks, once every 3-4 weeks, once every 4-5 weeks, once every 5-6 weeks, once every 6-7 weeks, once every 7-8 weeks and so forth. In other embodiments, the prophylactic effect of atovaquone can last at least a month and up to several months.

The present invention also provides methods for preventing a parasitic or fungal infection comprising the step of intramuscularly administering an effective amount of atovaquone to a subject. In a more specific embodiment, the atovaquone is present as a microparticle. The parasitic infection can be caused by Plasmodium, Toxoplasma or Babesiidae or the fungal infection can be caused by Pneumocystis. The atovaquone can be administered every few weeks including, but not limited to, every two weeks, every three weeks, every four weeks, every five weeks, every six weeks, every seven weeks, every 8 weeks and so forth. In a specific embodiment, atovaquone can be administered once every 2-8 weeks. In more specific embodiments, atovaquone can be administered once every 2-3 weeks, once every 3-4 weeks, once every 4-5 weeks, once every 5-6 weeks, once every 6-7 weeks, once every 7-8 weeks and so forth. In other embodiments, the prophylactic effect of atovaquone can last at least a month and up to several months.

The present invention also provides an intramuscularly-injectable formulation of a pharmaceutical composition comprising microparticle formulation of atovaquone, wherein the atovaquone is formulated for administration once every two to eight weeks. In further embodiments, the present invention provides a kit comprising a first container comprising the intramuscularly-injectable formulation described herein; a second container comprising a syringe; and instructions for injecting the formulation into a subject to prevent malaria. In one embodiment, the formulation comprises atovaquone already in suspension. In another embodiment, the formulation comprises a lyophilized powder. The kit can further comprise a sterile liquid excipient such as water.

The methods and compositions can also be used in combination with another anti-infective drug (or drugs) to prevent or treat malaria or another parasitic infection or a fungal infection. The parasitic infection can be caused by Plasmodium, Toxoplasma or Babesiidae or the fungal infection can be caused by Pneumocystis. In specific embodiments, the method further comprises administering, by the same or different route, another anti-malaria or anti-parasitic drug.

### BRIEF DESCRIPTION OF THE FIGURES

FIG. 1. Experimental design. Cohorts of mice were dosed with atovaquone or placebo on Day 0, and challenged with intravenous sporozoites at defined post-dose intervals (X). After challenge, blood samples were monitored (•) for appearance of parasites. Prophylaxis was considered successful if all animals in a dosing cohort remained parasite-free at 42 days post-challenge.

FIG. 2. Efficacy. In a dose-dependent manner intramuscular atovaquone provided successful malaria prophylaxis for up to 42 days post-challenge (filled circles: blue for diluted Mepron, yellow for washed Mepron). Prophylaxis failures are denoted by X (red for oral diluted Mepron, black for intramuscular diluted Mepron).

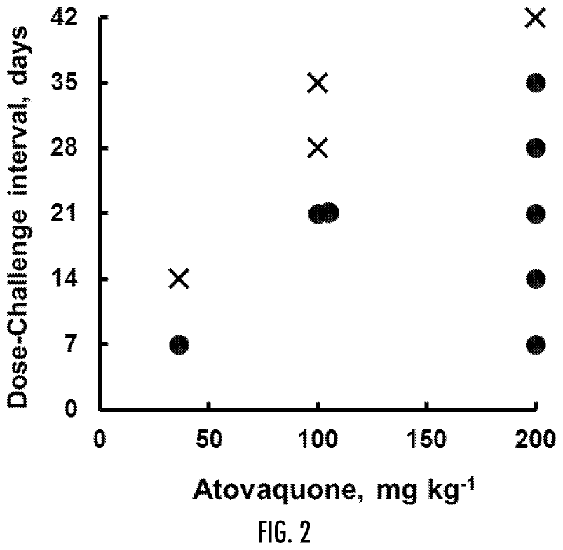

FIG. 3. Pharmacokinetic profile. Plasma concentrations of atovaquone in mice after intramuscular dose of 200 mg/kg. Half-life across the entire sampling interval is 7.6 days, substantially longer than the 9 hr half-life after oral dosing (Pudney et al., 6 J. TRAVEL MED. SUPPL. 1:S8-12 (1999). Insert, Plasma levels are dose proportional at 14 days post-dose.

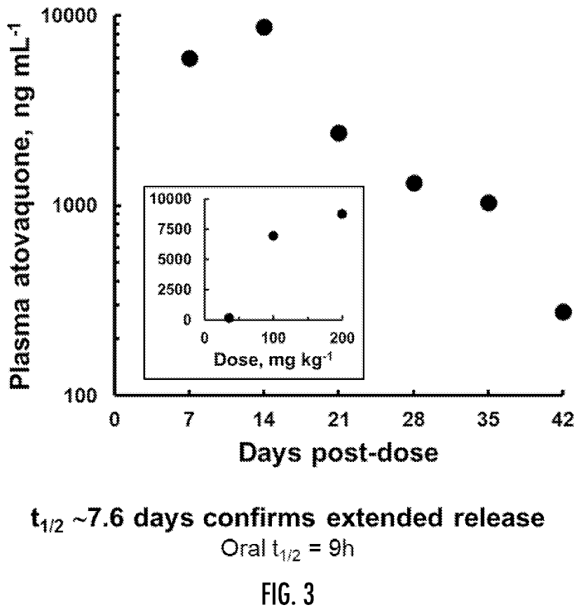

FIG. 4. Test for liver stage breakthrough. Animals dosed on day 0 were protected against day 7 challenges with 5,000, 50,000 or 500,000 sporozoites, indicating that liver stage protection is robust and not overwhelmed with a supra-lethal sporozoite challenge. Untreated animals succumbed within 9 days (X) of sporozoite challenges.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Atovaquone is a hydroxy-1,4-naphthoquinone and an antiprotozoal agent. It exists as a yellow crystalline solid that is practically insoluble in water, but is highly lipophilic. The structure of atovaquone is shown below:

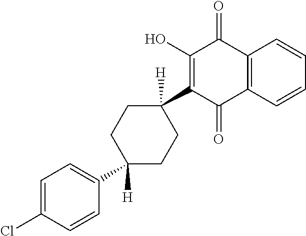 [SMILES: O=C1C(O)=C([C@H]2CC[C@H](c3ccc(Cl)cc3)CC2)C(=O)c2ccccc21]

The term “atovaquone” also includes pharmaceutically acceptable salts, solvates, stereoisomers and derivatives thereof, prodrugs thereof, as well as any polymorphic or amorphous forms thereof. Sold under the name Mepron®, atovaquone is sold in a suspension and is indicated for the prevention of Pneumocystis jiroveci pneumonia (PCP). The recommended oral liquid dosage is 1,500 mg (10 mL) once daily, administered with food.

For malaria, a combination preparation of atovaquone with proguanil hydrochloride is available under the trade name Malarone®, with a standard (adult) tablet containing 250 mg of atovaquone and 100 mg of proguanil hydrochloride. To prevent malaria in adults (weighing over 40 kg), one standard tablet should be taken once a day 24 to 48 hours before entering a malarial area, continuing with one tablet once a day for the duration of stay in the malarial area, and further continuing for 7 days after leaving the malarial area, i.e., 2.25 to 2.50 g of atovaquone for just a one-day stay in a malarial area. To treat malaria in an adult, four standard tablets should be taken every day for three days, i.e., 3 g of atovaquone in total.

The terms “patient,” “individual,” or “subject” are used interchangeably herein, and refer to a mammal, particularly, a human. The patient may be an individual in need of medication to prevent or prophylax a condition or, in other embodiments, in need of treatment.

It is to be appreciated that references to “preventing” or “prevention” relate to prophylactic treatment and includes preventing or delaying the appearance of clinical symptoms of the state, disorder or condition developing in a patient that may be afflicted with or predisposed to the state, disorder or condition but does not yet experience or display clinical or subclinical symptoms of the state, disorder or condition. As described herein, malaria can be prevented in a subject for a few weeks and additional injections of atovaquone can be administered. For example, atovaquone can be administered every few weeks including, but not limited to, every two weeks, every three weeks, every four weeks, every five weeks, every six weeks, every seven weeks, every 8 weeks and so forth. In a specific embodiment, atovaquone can be administered once every 2-8 weeks. In more specific embodiments, atovaquone can be administered once every 2-3 weeks, once every 3-4 weeks, once every 4-5 weeks, once every 5-6 weeks, once every 6-7 weeks, once every 7-8 weeks and so forth. In other embodiments, the prophylactic effect of atovaquone can last at least a month and up to several months.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject; (b) inhibiting the disease, i.e., arresting its development; and (c) relieving the disease, e.g., causing regression of the disease, e.g., to completely or partially remove symptoms of the disease. In a specific embodiment, the disease or condition is malaria. In particular embodiments, the disease is a parasitic or fungal infection.

As used herein, the term “effective,” means adequate to accomplish a desired, expected, or intended result. More particularly, an “effective amount” of a “prophylactically/therapeutically effective amount” is used interchangeably and refers to an amount of atovaquone necessary to provide the desired “prevention” or “treatment” or prophylactic/therapeutic effect, e.g., an amount that is effective to prevent, alleviate, treat or ameliorate symptoms of a disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “prophylactically/therapeutically effective amount” in any individual case can be determined by one of ordinary skill in the art by reference to the pertinent texts and literature and/or by using routine experimentation.

In particular embodiments, the atovaquone is provided as a microparticle. The term “microparticle” refers generally to a particle having a size of about 1 μm to about 1000 μm. As used herein, the term “microparticle,” in particular embodiments, has an average size of about 30 μm to about 1000 μm, about 100 μm to about 900 μm, about 200 μm to about 800 μm, or about 300 μm to about 700 μm, including ranges in between the foregoing. The microparticles of atovaquone can have an average particle size greater than 50 μm, greater than 100 μm, greater than 200 μm, greater than 300 μm, greater than 400 μm, greater than 500 μm, and so forth.

Moreover, in certain embodiments, the atovaquone of the present invention comprises a non-nanoparticle formulation. For example, the atovaquone of the present invention is not provided (1) as a solid composition comprising nanoparticles of atovaquone dispersed within one or more carrier materials; (2) a plurality of nanoparticles of atovaquone dispersed in an aqueous medium, each nanoparticle of atovaquone being a core around at least some of which an outer layer composed of one or more carrier materials; or (3) as an oily dispersion, comprising a plurality of nanoparticles of atovaquone and one or more carrier materials dispersed in an oily medium. The term “nanoparticle” generally to a particle having a size of about 1 nm to 1000 nm.

In accordance with an embodiment, the present invention provides compositions and methods for the prophylaxis of malaria. The present invention can also be used to prevent other parastitic and fungal diseases in a subject including, but not limited to, toxoplasmosis (caused by the parasite Toxoplasma gondii), babesiosis (caused by the parasite Babesia microti) and Pneumocystis pneumonia (caused by the fungus Pneumocystis jirovecii). In further embodiments, the compositions and methods of the present invention can be used to treat malaria or another parasitic infection or a fungal infection. Such embodiments include the atovaquone formulation described herein in combination with another anti-malaria or anti-parasitic drug (by the same or a different route than the intramuscular injection of the atovaquone formulation). More specifically, the present invention provides compositions and methods for prophylaxis and/or treatment of a parasitic infection in a subject, comprising administering to the subject a pharmaceutical composition comprising an intramuscular formulation of atovaquone. Optionally, other drugs (including another anti-parasitic compound) can be administered by the same or different route. Such drugs refer to the following (non-exhaustive) list of other drugs that may be used in combination with atovaquone formulated in accordance with the invention in a combination prophylactic and/or treatment therapy: proguanil, mefloquine, chloroquine, hydroxychloroquine, quinine, quinidine, artemether, lumefantrine, primaquine, doxycycline, tetracycline, clindamycin, dihydroartemisinin, piperaquine, and pyrimethamine with or without sulfadoxine, as well as pharmaceutically acceptable salts, solvates and derivatives thereof, prodrugs thereof, and any polymorphic or amorphous forms thereof. The present invention may also be combined with one or more experimental anti-malarials under development including, but not limited to, those in the Medicines for Malaria Venture portfolio: DDD498, PA92, MMV253, GSK030, DSM421, AN13762, UCT943, P218, SJ733, ferroquine, KAF156, cipargamin, DSM265, tafenoquine, pyronaridine, amodiaquine.

As used herein, the term “anti-parasitic compound” means one or more active agents from the class of drugs known as antiprotozoals, anthelmintics, ectoparasiticides, and similar compounds. These groups include antiamebiasis agents, antifascioliasis agents, antifiliariasis agents, antileshmaniasis agents, antimalarials, antischistosomal agents, antitapeworm agents and antitrypanosomiasis agents. Examples of such compounds include arsenicals, benzamidine, napthalenesulfonate, nitroimidazole, macrolides, nitrofuran, pentavalent antimonials, phosphoryl choline, neomycin, thiazole, aminoacridine, oxyquinoline, tetracycline, trimethoprim/sulfamethoxazole, pyirmethamine, aminoquinolines, 4-methanolquinolines, biguanides, sulfonamides, sesquiterpene lactones, atovaquone, pyronaridine, piperaquine, artesunate-amodiaquine, nitroimidazole derivatives, ivermectin, and related compounds. Also included in the term “anti-parasitic compounds” are vaccines and antibodies to infectious parasites.

Embodiments of the invention also include a process for preparing pharmaceutical products comprising the compound(s). The term “pharmaceutical product” means a composition suitable for pharmaceutical use (pharmaceutical composition), as defined herein. Pharmaceutical compositions formulated for particular applications comprising the compounds of the present invention are also part of this invention, and are to be considered an embodiment thereof.

Furthermore, the compositions and methods of the present invention can include pharmaceutical compositions consisting essentially of atovaquone. In a specific embodiment, a pharmaceutical composition consists of atovaquone and a pharmaceutically acceptable carrier. In other embodiments, a pharmaceutical composition comprises atovaquone and no other drug or active ingredient.

With respect to pharmaceutical compositions described herein, the pharmaceutically acceptable carrier can be any of those conventionally used and generally regarded by the FDA as safe (GRAS) for intramuscular injection, and is limited only by physico-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. Examples of the pharmaceutically acceptable carriers include soluble carriers such as known buffers which can be physiologically acceptable (e.g., phosphate buffer). It is preferred that the pharmaceutically acceptable carrier be one which is chemically inert to the active agent(s), and one which has little or no detrimental side effects or toxicity under the conditions of use.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The choice of carrier will be determined, in part, by the particular compound, as well as by the particular method used to administer the compound. Accordingly, there are a variety of suitable formulations of the pharmaceutical composition of the invention. In particular embodiments, atovaquone is formulated for intramuscular injection. In other embodiments, formulations for parenteral, subcutaneous, intravenous, intraarterial, intrathecal and interperitoneal administration are contemplated, and are in no way limiting. More than one route can be used to administer the compound(s), and in certain instances, a particular route can provide a more immediate and more effective response than another route.

The parenteral formulations will typically contain from about 0.5% to about 25% by weight of the compounds in suspension. Preservatives and buffers may be used. In order to minimize or eliminate irritation at the site of injection, such compositions may contain one or more nonionic surfactants, for example, having a hydrophile-lipophile balance (HLB) of from about 12 to about 17. The quantity of surfactant in such formulations will typically range from about 5% to about 15% by weight. Suitable surfactants include, for example, polyethylene glycol sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

For purposes of the invention, the amount or dose of atovaquone administered should be sufficient to effect a prophylactic or therapeutic response in the subject over a reasonable time frame. The dose will be determined by the efficacy of the particular compound and the condition of a human, as well as the body weight of a human to be treated. The dose of atovaquone of the present invention also will be determined by the existence, nature and extent of any adverse side effects that might accompany the administration of a particular compound. Typically, an attending physician will decide the dosage of the compound with which to treat each individual patient, taking into consideration a variety of factors, such as age, body weight, general health, diet, sex, compound to be administered, route of administration, and the severity of the condition being treated. In particular embodiments, the atovaquone comprises a dosage in an amount effective to prevent malaria for between two to five weeks as described herein. Subject can receive follow up intramuscular injections of atovaquone to further prevent malaria for additional time periods as necessary. In non-limiting, specific embodiments, an intramuscular dose of atovaquone comprises up to 35 mg/kg in 10 mL injection (25% suspension, 2×5 ml injections). In another embodiment, the intramuscular dose of atovaquone comprises 1 ml injection. The injection can also comprise about 1-4 ml. In certain embodiments, the dose of atovaquone can comprise about 350 μg to about 35 mg/kg, e.g., 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1 mg/kg, 5 mg/kg, 10 mg/kg, 15 mg/kg, 20 mg/kg, 25 mg/kg, 30 mg/kg, and 35 mg/kg. Range can include 350 μg to 35 mg/kg and any range in between including, but not limited to, 400 μg to 35 mg/kg, 450 μg to 35 mg/kg, 500 μg to 35 mg/kg. 550 μg to 35 mg/kg, 600 μg to 35 mg/kg, 650 μg to 35 mg/kg, 700 μg to 35 mg/kg, 750 μg to 35 mg/kg, 800 μg to 35 mg/kg, 850 μg to 35 mg/kg, 900 μg to 35 mg/kg, 950 μg to 35 mg/kg, 1 mg/kg to 35 mg/kg, 5 mg/kg to 35 mg/kg, 10 mg/kg to 35 mg/kg, 15 mg/kg to 35 mg/kg, 20 mg/kg to 35 mg/kg, 25 mg/kg to 30 mg/kg, and 30 mg/kg to 35 mg/kg.

Without further elaboration, it is believed that one skilled in the art, using the preceding description, can utilize the present invention to the fullest extent. The following examples are illustrative only, and not limiting of the remainder of the disclosure in any way whatsoever.

### Examples

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Sporozoite Parasites for Challenge.

Four- to six-day old Anopheles stephensi adult mosquitoes fasted for 6 h were fed on Swiss Webster mice infected with P. berghei (ANKA 2.34 strain). Fed mosquitoes were maintained for 18 d (19° C., 80% relative humidity), when salivary glands were harvested into RPMI medium and disrupted by several passages through a 0.5 in 28-gauge needle to release sporozoites. Parasites were counted by hemocytometer and diluted to 25,000 mL⁻¹ RPMI. C57BL6 mice were challenged by intravenous infusion of 5,000 sporozoites in 200 μL RPMI.

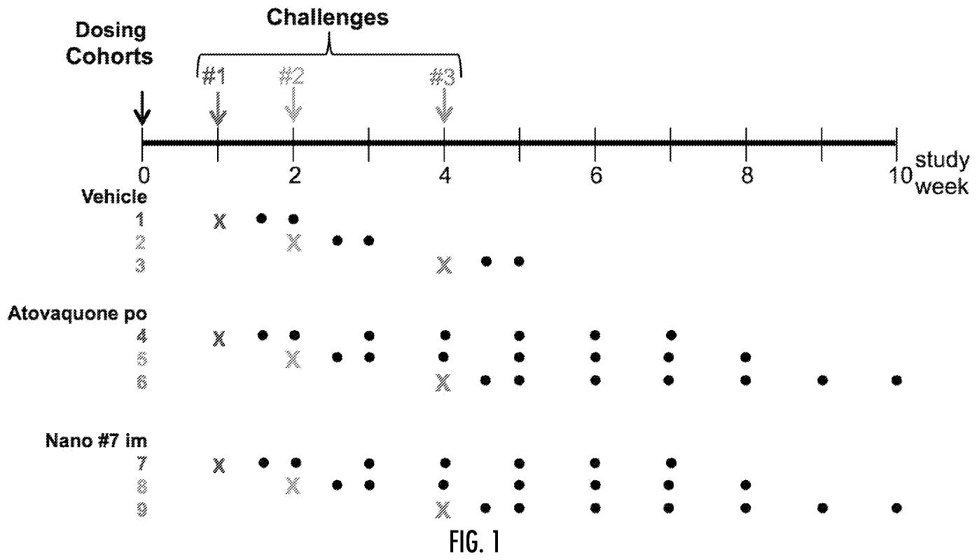

In Vivo Pharmacokinetics and Efficacy (See Schema in FIG. 1).

All dosing materials were constituted just before use. Mepron suspension (150 mg/mL atovaquone) was the starting material. Pluronic F-68 (10%) was used to dilute Mepron to required dosing concentration (≤100 mg/mL), or as vehicle control. For the washed drug preparation, atovaquone was pelleted from diluted Mepron (50 mg/mL, 13,000×g, 10 min) and the supernatant was replaced with equal volume 0.9% NaCl. Washing was repeated three times prior to final resuspension of excipient-depleted atovaquone at 50 mg/mL in 0.9% NaCl for dosing. C57BL6 male mice (˜20 g; 3-5 per group) were injected intramuscularly (biceps femoris; 23-gauge needle, 100 μl Hamilton syringe) with diluted or washed Mepron (40 μl total volume, 20 μl per limb). Oral Mepron was administered by gavage. At predetermined post-dose intervals, animals were challenged with P. berghei ANKA by infusion of 5,000 sporozoites into a tail vein. Starting 4 d post-challenge then weekly thereafter for 42 d, tail snip blood samples were examined for parasites via Giemsa-stained thin smears. Each experiment included an untreated cohort to validate the challenge. Efficacy outcomes were binary. The appearance of parasitemia in any animal in a cohort was designated a dosing failure. Animals remaining parasite-free at 42 days post-challenge were considered protected, and treatment was deemed protective if all animals in a dosing cohort were protected. To obtain atovaquone pharmacokinetics, blood was harvested (microtainer tubes, BD Biosciences), centrifuged (1300×g, 10 min, 4° C.), and plasma was collected and stored at −80° C. until use. Atovaquone concentration in plasma was assayed by UPLC-MS/MS as described (Chambliss et al., 1 J. APPLIED LAB. MED. 400-09 (2017).

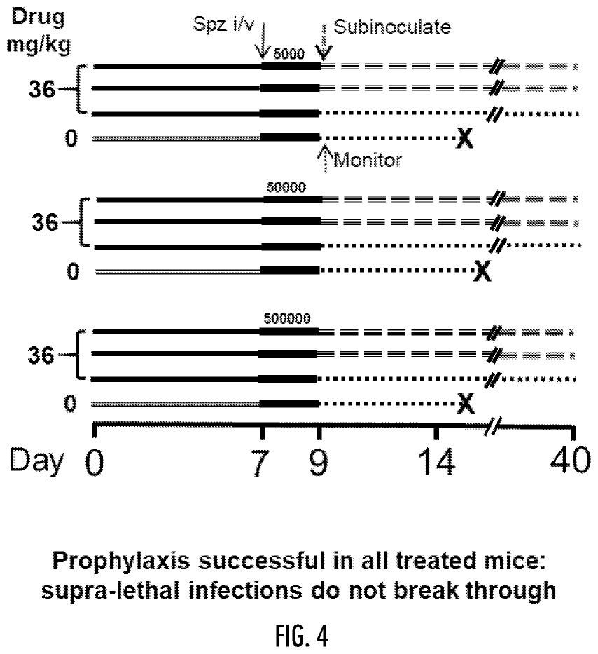

Test for Liver Stage Breakthrough (See Schema in FIG. 4).

Mepron (150 mg/ml atovaquone) was diluted to 18 mg/ml with 10% Pluronic F-68. C57/BL6 mice were injected with a dose of 36 mg/kg (see in vivo efficacy section above for details). 7 days post-dose, three groups of 4 mice each (3 dosed and 1 untreated per group) were challenged with intravenous infusions of 5000, 50000, or 500000 sporozoites. 48-51 h post-challenge, two dosed mice from each treated and challenged group were exsanguinated and their blood was subinoculated into naïve mice to allow liver-emergent parasites to proliferate in a drug-free background. All mice were monitored periodically for emergence of blood-stage parasites.

1. A method for preventing malaria comprising the step of intramuscularly administering an effective amount of atovaquone to a subject.

2. The method of claim 1, wherein the atovaquone is present as a microparticle.

3. The method of claim 1, wherein the atovaquone is administered once every two to eight weeks.

4. A method for preventing a parasitic or fungal infection comprising the step of intramuscularly administering an effective amount of atovaquone to a subject.

5. The method of claim 1, wherein the atovaquone is present as a microparticle.

6. The method of claim 1, wherein the atovaquone is administered once every two to eight weeks.

7. The method of claim 4, wherein the parasitic infection is caused by Plasmodium, Toxoplasma or Babesiidae or wherein the fungal infection is caused by Pneumocystis.

8. An intramuscularly-injectable formulation of a pharmaceutical composition comprising a microparticle formulation of atovaquone, wherein the atovaquone is formulated for administration once every two to eight weeks.

9. A kit comprising a first container comprising the intramuscularly-injectable formulation of claim 8; a second container comprising a syringe; and instructions for injecting the formulation into a patient to prevent malaria.

10. The kit of claim 9, wherein the formulation is a lyophilized powder.

11. The kit of claim 10, further comprising a sterile liquid excipient.
